(19) United States
(12) Patent Application Publication
(10) Pub. No.: US 2023/0201585 A1
(43) Pub. Date: Jun. 29, 2023

(54) IMPEDANCE MONITORING METHOD AND NON-IMPLANTABLE ELECTRICAL STIMULATION DEVICE

(71) Applicant: GIMER MEDICAL. Co. LTD., New Taipei City (TW)

(72) Inventors: Wan-Ting CHIANG, New Taipei City (TW); Jian-Hao PAN, New Taipei City (TW)

(73) Assignee: GIMER MEDICAL. Co. LTD., New Taipei City (TW)

(21) Appl. No.: 17/981,643

(22) Filed: Nov. 7, 2022

(30) Foreign Application Priority Data
Dec. 29, 2021 (CN) 202111638877.X

(51) Int. Cl. A61N 1/36; A61B 5/0531; A61N 1/04; A61N 1/08

(52) U.S. Cl. CPC A61N 1/36031; A61B 5/0531; A61N 1/0456; A61N 1/08; A61N 1/36034

(57) ABSTRACT

An impedance monitoring method is applied to a non-implantable electrical stimulation device including an electrical stimulator and an electrode assembly. The electrical stimulator is detachably electrically connected to the electrode assembly, and stores the impedance values of the electrical stimulator and the electrode assembly. The impedance monitoring method includes the following steps. The electrical stimulator generates an electrical stimulation signal. The electrical stimulation signal performs electrical stimulation of a target area through the electrode assembly. The electrical stimulator samples the electrical stimulation signal to calculate the total impedance value corresponding to the electrical stimulation signal. The electrical stimulator calculates the tissue impedance value according to the total impedance value, the impedance value of the electrical stimulator, and the impedance value of the electrode assembly. The tissue impedance value is used to calculate the energy value corresponding to the electrical stimulation signal transmitted to the target area.

### CROSS REFERENCE TO RELATED APPLICATIONS

This application claims priority of China Patent Application No. 202111638877.X, filed on Dec. 29, 2021, the entirety of which is incorporated by reference herein.

### BACKGROUND

### Technology Field

The disclosure relates to an electrical stimulation technology.

### Description of the Related Art

In recent years, dozens of therapeutic nerve electrical stimulation devices have been developed, and at least tens of thousands of people undergo electrical stimulation device implantation every year. Due to the development of precision manufacturing technology, the size of medical devices has been miniaturized and may be implanted inside the human body, for example, an implantable electrical stimulation device.

After an implantable electrical stimulation device is implanted into the human body, the human tissue may gradually build up and coat the implantable electrical stimulation device, causing the tissue's impedance to change and leading to poor electrical stimulation. Therefore, how to effectively monitor tissue impedance has become an important issue.

### SUMMARY

An impedance monitoring method and a non-implantable electrical stimulation device are provided by the embodiment of the disclosure to overcome the problems mentioned above.

An embodiment of the disclosure provides an impedance monitoring method. The impedance monitoring method is applied to a non-implantable electrical stimulation device. The non-implantable electrical stimulation device includes an electrical stimulator and an electrode assembly. The electrical stimulator is detachably electrically connected to the electrode assembly. The electrical stimulator stores the impedance value of the electrical stimulator and the impedance value of the electrode assembly. The impedance monitoring method includes the following steps. The electrical stimulator is used to generate an electrical stimulation signal. The electrical stimulation signal performs electrical stimulation of a target area through the electrode assembly. The electrical stimulator is used to sample the electrical stimulation signal to calculate the total impedance value corresponding to the electrical stimulation signal. The electrical stimulator is used to calculate the tissue impedance value according to the total impedance value, the impedance value of the electrical stimulator, and the impedance value of the electrode assembly. The tissue impedance value is used to calculate the energy value corresponding to the electrical stimulation signal transmitted to the target area.

An embodiment of the disclosure provides a non-implantable electrical stimulation device. The non-implantable electrical stimulation device includes an electrode assembly and an electrical stimulator. The electrical stimulator is detachably electrically connected to the electrode assembly. The electrical stimulator includes a storage unit, an electrical stimulation signal generating circuit, a sampling module and a calculation module. The storage unit is configured to store the impedance value of the electrical stimulator and the impedance value of the electrode assembly. The electrical stimulation signal generating circuit is configured to generate an electrical stimulation signal, and to use the electrical stimulation signal to perform electrical stimulation of a target area. The sampling module is configured to sample the electrical stimulation signal. The calculation module is configured to calculate the total impedance value corresponding to the electrical stimulation signal according to the sampled electrical stimulation signal, and to calculate the tissue impedance value according to the total impedance value, the impedance value of the electrical stimulator and the impedance value of the electrode assembly. The tissue impedance value is used to calculate the energy value corresponding to the electrical stimulation signal transmitted to the target area.

Other aspects and features of the disclosure will become apparent to those with ordinary skill in the art upon review of the following descriptions of specific embodiments of the impedance monitoring method and the non-implantable electrical stimulation device provided by the embodiment.

### BRIEF DESCRIPTION OF THE DRAWINGS

The disclosure can be more fully understood by reading the subsequent detailed description and examples with references made to the accompanying drawings, wherein:

FIG. 1A is a perspective view of a non-implantable electrical stimulation device according to an embodiment of the disclosure;

FIG. 1B is a perspective view of a non-implantable electrical stimulation device shown in FIG. 1A form another angle;

FIG. 1C is an exploded schematic view of a non-implantable electrical stimulation device shown in FIG. 1A;

FIG. 2 is a block diagram of a non-implantable electrical stimulation device according to an embodiment of the disclosure;

FIG. 3 is a waveform diagram of an electrical stimulation signal of a non-implantable electrical stimulation device according to an embodiment of the disclosure;

FIG. 4 is a schematic view of a non-implantable electrical stimulation device according to an embodiment of the disclosure;

FIG. 5 is block diagram of a control unit according to an embodiment of the disclosure;

FIG. 6 is block diagram of an impedance compensation device according to an embodiment of the disclosure;

FIG. 7 is a schematic view of an impedance compensation model according to an embodiment of the disclosure;

FIG. 8 is a flowchart of an impedance monitoring method according to an embodiment of the disclosure;

FIG. 9 is a flowchart of an impedance monitoring method according to another embodiment of the disclosure;

FIG. 10 is a flowchart of a processing method of an electrical stimulation signal according to an embodiment of the disclosure;

FIG. 11 is a flowchart of an updating method of an output tissue impedance average value according to an embodiment of the disclosure; and

FIG. 12 is a flowchart of an adjustment method of an output current according to an embodiment of the disclosure.

### DETAILED DESCRIPTION OF THE DISCLOSURE

The following description is of the contemplated mode of carrying out the disclosure. This description is made for the purpose of illustrating the general principles of the disclosure and should not be taken in a limiting sense. The scope of the disclosure is determined by reference to the appended claims.

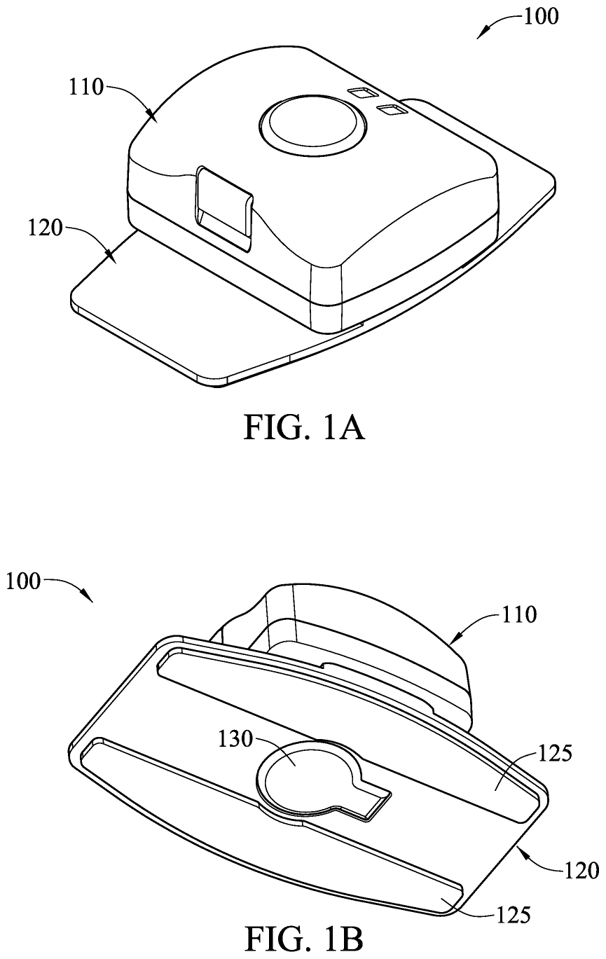

FIG. 1A is a perspective view of a non-implantable electrical stimulation device according to an embodiment of the disclosure. FIG. 1B is a perspective view of a non-implantable electrical stimulation device shown in FIG. 1A form another angle. FIG. 1C is an exploded schematic view of a non-implantable electrical stimulation device shown in FIG. 1A. Please refer to FIG. 1A, FIG. 1B and FIG. 1C. The non-implantable electrical stimulation device 100 includes an electrical stimulator 110 and an electrode assembly 120. In the embodiment, the non-implantable electrical stimulation 100 is, for example, a transcutaneous electrical nerve stimulation device (TENS device), which does not necessarily need to be implanted in the body or subcutaneously, but is directly attached to the body surface or skin of a living body through the electrode assembly 120 for electrical stimulation of a target area. In the embodiment, the living body is, for example, the body of a user or a patient. The target area includes the body surface or skin of the living body, and the target area is, for example, a superficial nerve within 10 millimeters (mm) from the body surface to relieve pain or other symptoms of disease. In addition, the main difference between the non-implantable electrical stimulation device 100 of the embodiment and the general muscle electrical stimulation device is that the target area for electrical stimulation performed by the non-implantable electrical stimulation device 100 of the embodiment is the nerve, rather than the muscle. Therefore, when the non-implantable electrical stimulation device 100 performs an electrical stimulation, the distance between the two electrodes of the electrode assembly 120 is relatively close, and the distance between the two adjacent electrodes is, for example, between 5 mm and 35 mm. The aforementioned two electrodes may be positive and negative electrodes, or one working electrode and another reference electrode, wherein the working electrode sends an electrical stimulation signal, and the reference electrode send a voltage signal at a DC fixed level.

In the embodiment, the electrical stimulator 110 is disposed on the upper half of the non-implantable electrical stimulation device 100. The electrical stimulator 110 includes a casing 111, a circuit board 112, at least two first electrical connectors 113 and at least one first magnetic unit 114.

The casing 111 includes an upper casing 111a and a lower casing 111b. The upper casing 111a and the lower casing 111b are combined to form an accommodating space. Most of the components required for the electrical stimulator 110 are disposed in the accommodating space, including the circuit board 112, the first electrical connectors 113, the first magnetic unit 114, and other components.

On the other hand, the electrode assembly 120 is disposed in the lower half of the non-implantable electrical stimulation device 100 and is connected with the lower casing 111b under of the electrical stimulator 110. The electrode assembly 120 includes a body 121, two electrodes 122, at least one second magnetic unit 123, at least two second electrical connectors 124 and a conductive gel 125. The electrical stimulator 110 may electrically transmit the sent electrical stimulation signal from the circuit board 112 to electrodes (i.e., the electrodes 122) of other components, such that the non-implantable electrical stimulation device 100 may perform electrical stimulation on the target area of the living body.

In the embodiment, the body 121 of the electrode assembly 120 has certain flexibility, such that it may be easily attached to different parts of the living body, and the material of the body 121 of the electrode assembly 120 may be rubber, silicone or other flexible materials.

In the embodiment, the electrode assembly 120 may be a magnetic electrode assembly. In addition, the above two electrodes 122 may be thin film electrodes. Furthermore, the above electrodes 122 are printed or sprayed on a surface F1 of the body 121 opposite to the casing 111 by a conductive material (i.e., silver paste), and the thickness of the above electrodes 122 may be 0.01 mm to 0.30 mm. The surface F1 is the lower surface of the body 121 shown in FIG. 1C, and is also a side facing the using part of the user during use.

In some embodiments, when using the non-implantable electrical stimulation device 100 of the embodiment, the conductive gel 125 of the electrode assembly 120 may be coated on the lower surface of the body 121. In some embodiments, the conductive gel 125 may be disposed on a sticking surface of the electrode 122 away from the body 121, and one electrode 122 may be correspondingly disposed with the conductive gel 125. The conductive gel 125 is not only sticky so that the electrode patch provided with the electrodes may be attached to the body surface or skin of the living body, but also reduces the contact resistance between the electrodes 122 and the body surface or skin of the living body due to the arrangement of the conductive gel 125, and may make the current of the electrodes evenly spread over the entire attached body surface area, avoiding the stinging sensation of the living body. At the same time, the comfort of using the non-implantable electrical stimulation device 100 is increased. That is, the electrode assembly 120 of the embodiment does not have a lead type, and the electrode assembly 120 may be two thin film electrodes 122 combined with the conductive gel 125 for electrical stimulation.

In addition, the first magnetic unit 114 of the electrical stimulator 110 is disposed in the accommodating space, for example, between the circuit board 112 and the casing 111. It should be noted that the first magnetic unit 114 in the embodiment is disposed under the circuit board 112.

In the non-implantable electrical stimulation device 100 of the embodiment, the electrical stimulator 110 includes at least one first magnetic unit 114, the electrode assembly 120 includes at least one second magnetic unit 123, and the numbers of the first magnetic unit 114 and the second magnetic unit 123 may be the same or different. The embodiment is described by taking as an example that four first magnetic units 114 correspond to four magnetic units 123. In addition, the electrode assembly 120 is detachably positioned on one side of the electrical stimulator 110 (i.e., one side of the lower casing 111b of the electrical stimulator 110) by being adsorbed by the at least one first magnetic unit 114 and the at least one second magnetic unit 123.

In addition, in the embodiment, the lower casing 111b of the electrical stimulator 110 may be correspondingly designed to have a protruding configuration 130 (as shown in FIG. 1B) at a position corresponding to the opening 126 of the body 121. After the electrode assembly 120 is assembled to the electrical stimulator 110, the protruding configuration 130 of the lower casing 111b protrudes from the opening 126 of the body 121. Therefore, the electrode assembly 120 may be more stably disposed on the electrical stimulator 110, and the alignment of the electrode assembly 120 and the electrical stimulator 110 is facilitated.

After the electrical stimulation signal is sent from the circuit board 112, the electrical stimulator 110 may be electrically connected to electrodes 122 through the first electrical connectors 113 and the second electrical connectors 124 (male rivets 124b and female rivets 124a) in sequence, and finally the electrical stimulation signal electrically stimulates the target area through the conductive gel 125 disposed corresponding to the electrodes 122. In the embodiment, in addition to above components, the non-implantable electrical stimulation device 100 is provided with a battery 115 or a power module in the accommodating space of the electrical stimulator 110, and the battery 115 or the power module may output power to the circuit board 112.

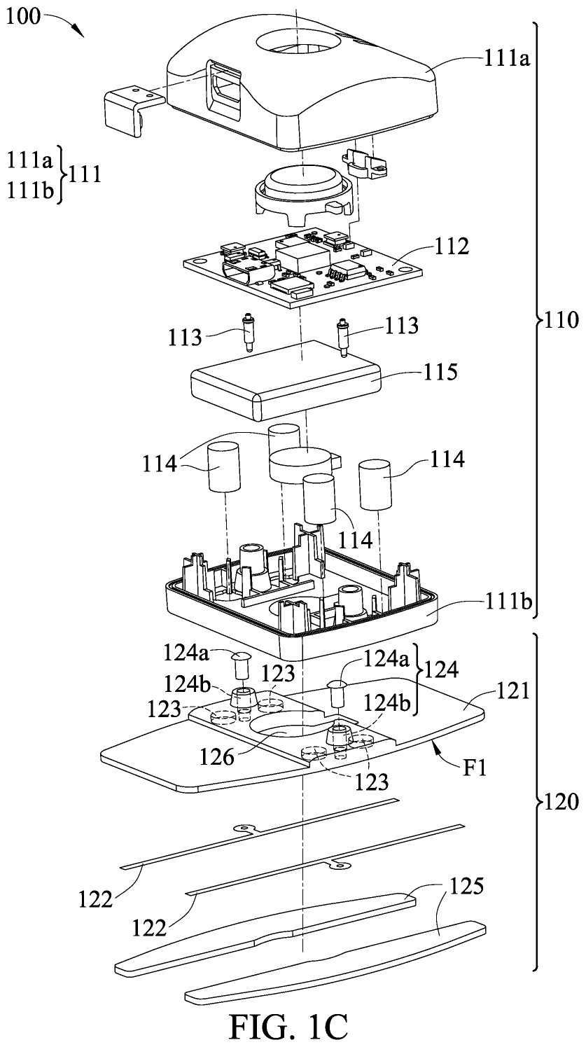

FIG. 2 is a block diagram of a non-implantable electrical stimulation device 100 according to an embodiment of the disclosure. As shown in FIG. 2, the non-implantable electrical stimulation device 100 may at least include a power management circuit 210, an electrical stimulation signal generating circuit 220, a measurement circuit 230, a control unit 240, a communication circuit 250 and a storage device 260. In addition, the electrical stimulation signal generating circuit 220, the measurement circuit 230, the control unit 240, the communication circuit 250 and the storage device 260 may be disposed on the circuit board 112 of the electrical stimulator 110 shown in FIG. 1C. It should be noted that the block diagram shown in FIG. 2 is only for the convenience of explaining the embodiment of the disclosure, but the disclosure is not limited to FIG. 2. The non-implantable electrical stimulation device 100 may also include other components.

According to an embodiment of the disclosure, the non-implantable electrical stimulation device 100 may be electrically coupled to an external control device 200. The external control device 200 may include an operation interface. According to the operation of the user on the operation interface, the external control device 200 may generate a command or a signal to be transmitted to the non-implantable electrical stimulation device 100, and transmit the command or the signal to the non-implantable electrical stimulation device 100 through a manner of a wired communication (i.e., a transmission line). According to an embodiment of the disclosure, the external control device 200 may be a smart phone, but the disclosure is not limited thereto.

Furthermore, according to another embodiment of the disclosure, the external control device 200 may also transmit the command or the signal to the non-implantable electrical stimulation device 100 through a manner of a wireless communication (i.e., Bluetooth, Wi-Fi, or NFC, but the disclosure is not limited thereto).

According to an embodiment of the disclosure, the non-implantable electrical stimulation device 100 and the external control device 200 may be integrated into one device. According to an embodiment of the disclosure, the non-implantable electrical stimulation device 100 may be an electrical stimulation device with the battery 115, or an electrical stimulation device with the power wirelessly transmitted by the external control device 200.

According to an embodiment of the disclosure, the power management circuit 210 is configured to provide the power to the internal components and circuits in the non-implantable electrical stimulation device 100. The power provided by the power management circuit 210 may be from a built-in rechargeable battery (i.e., the battery 115) or the external control device 200, but the disclosure is not limited thereto. The external control device 200 may provide the power to the power management circuit 210 through a wireless power supply technology. The power management circuit 210 may be activated or deactivated according to the command of the external control device 200. According to an embodiment of the disclosure, the power management circuit 210 may include a switch circuit (not shown). The switch circuit may be turned on or off according to the command of the external control device 200 to activate or deactivate the power management circuit 210.

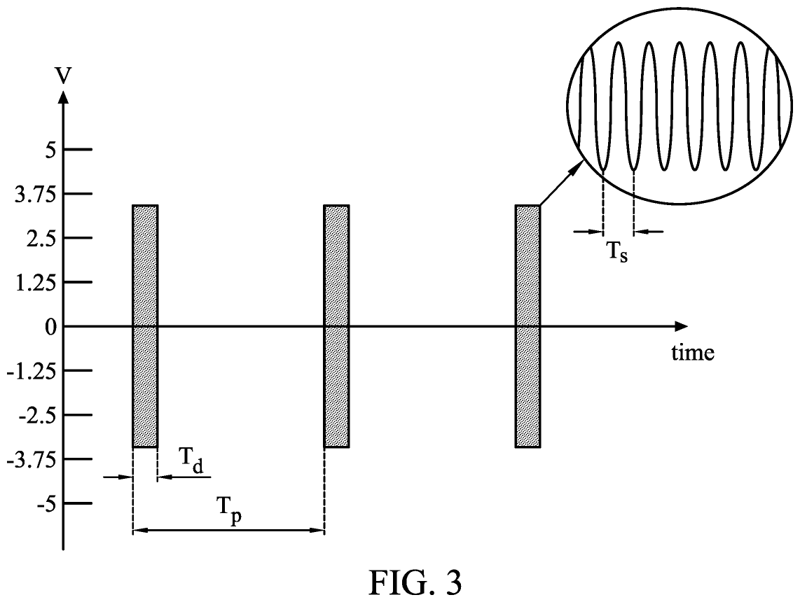

According to an embodiment of the disclosure, the electrical stimulation signal generating circuit 220 is configured to generate the electrical stimulation signal. The electrical stimulation signal generating circuit 220 may transmit the generated electrical stimulation signal to the electrodes 122 of the electrode assembly 122 through the first electrical connectors 113 and the second electrical connectors 124, so as to electrically stimulate the target area of the living body (i.e., a human or an animal) through the conductive gel 125 disposed corresponding to the electrodes 122. The above target area is, for example, a median nerve, a tibial nerve, a vagus nerve, a trigeminal nerve or other superficial nerves, but the disclosure is not limited thereto. The detailed structure of the electrical stimulation signal generating circuit 220 will be described with reference to FIG. 4.

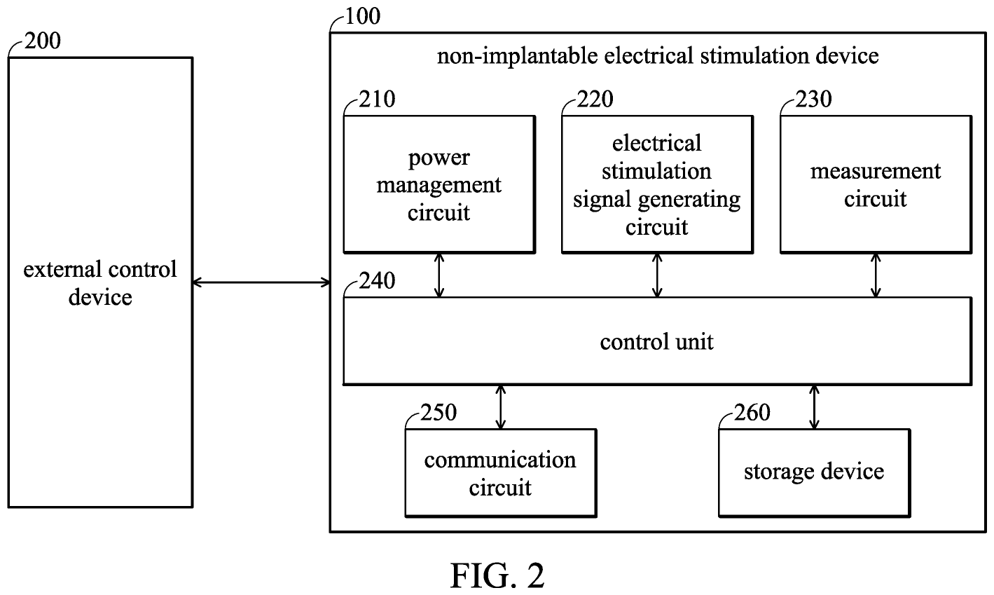

FIG. 3 is a waveform diagram of an electrical stimulation signal of a non-implantable electrical stimulation device according to an embodiment of the disclosure. As shown in FIG. 3, according to an embodiment of the disclosure, the above electrical stimulation signal may be a pulsed radio-frequency (PRF) signal (or referred to as a pulse signal), a continuous sine wave, a continuous triangular wave, etc., but the embodiment of the disclosure is not limited thereto. In addition, when the electrical stimulation signal is a pulse alternating signal, one pulse cycle time Tₚ includes one pulse signal and at least one rest period of time, and the pulse cycle time Tₚ is the reciprocal of the pulse repetition frequency. The pulse repetition frequency range (also referred to as the pulse frequency range) is, for example, between 0 and 1 KHz, preferably between 1 and 100 Hz. In the embodiment, the pulse repetition frequency of the electrical stimulation signal is, for example, 2 Hz. In addition, the duration time T_{d} of the pulse (i.e., a pulse width) in one pulse cycle time is, for example, between 1 and ˜250 milliseconds (ms), preferably between 10 and 100 ms. In the embodiment, the duration time T_{d} is, for example, 25 ms. In the embodiment, the frequency of the electrical stimulation signal is 500 KHz, in other words, the cycle time Tₛ of the electrical stimulation signal is about 2 microseconds (μs). Furthermore, the frequency of the above electrical stimulation signal is the intra-pulse frequency in each pulse alternating signal of FIG. 3. In some embodiments, the above intra-pulse frequency range of the above electrical stimulation signal is, for example, 1 KHz to 1000 KHz. It should be noted that in each of the embodiments of the disclosure, if only the frequency of the electrical stimulation signal is described, it refers to the intra-pulse frequency of the electrical stimulation signal. In some embodiments, the intra-pulse frequency range of the above electrical stimulation signal is, for example, from 200 KHz to 800 KHz. In some embodiments, the intra-pulse frequency range of the above electrical stimulation signal is, for example, from 480 KHz to 520 KHz. In some embodiments, the intra-pulse frequency of the above electrical stimulation signal is, for example, 500 KHz. The voltage range of the above electrical stimulation signal may be between −25 V and +25 V. Furthermore, the voltage range of the above electrical stimulation signal may further be between −20V and +20V. The current range of the above electrical stimulation signal may be between 0 and 60 mA. Furthermore, the current range of the above electrical stimulation signal may further be between 0 and 50 mA.

According to an embodiment of the disclosure, the user may operate the non-implantable electrical stimulation device 100 for electrical stimulation only when the user fells the need (for example, the symptoms become severe or not relieved). After the non-implantable electrical stimulation device 100 performs one electrical stimulation on the target area, the non-implantable electrical stimulation device 100 needs to wait a limited time before performing the next electrical stimulation on the target area. For example, after the non-implantable electrical stimulation device 100 performs one electrical stimulation on the target area, the non-implantable electrical stimulation device 100 needs to wait for 30 minutes (i.e., the limited time) before performing the next electrical stimulation on the target area, but the disclosure is not limited thereto. The limited time may also be any time interval within 45 minutes, 1 hour, 4 hours or 24 hours.

According to an embodiment of the disclosure, the measurement circuit 230 may measure the voltage value and the current value of the electrical stimulation signal according to the electrical stimulation signal generated by the electrical stimulation signal generating circuit 220. Furthermore, the measurement circuit 230 may measure the voltage value and the current value on the tissue of the target area of the living body (i.e., the body of the user or patient). According to an embodiment of the disclosure, the measurement circuit 230 may adjust the current and the voltage of the electrical stimulation signal according to the instruction of the control unit 240. The detailed structure of the measurement circuit 230 will be described with reference to FIG. 4.

According to an embodiment of the disclosure, the control unit 240 may be a controller, a microcontroller or a processor, but the disclosure is not limited thereto. The control unit 240 may be configured to control the electrical stimulation signal generating circuit 220 and the measurement circuit 230. The operation of the control unit 240 will be described below with reference to FIG. 4.

According to an embodiment of the disclosure, the communication circuit 250 may be configured to communicate with the external control device 200. The communication circuit 250 may transmit the command or the signal received from the external control device 200 to the control unit 240, and transmit the data measured by the non-implantable electrical stimulation device 100 to the external control device 200. According to an embodiment of the disclosure, the communication circuit 250 may communicate with the external control device 200 in a wireless or a wired communication manner.

According to an embodiment of the disclosure, when performing the electrical stimulation, all of the electrodes of the non-implantable electrical stimulation device 100 are activated or enabled. Therefore, the user does not need to select which electrodes on the electrode assembly 120 need to be activated, and which activated electrode is negative or positive polarity.

Compared with the traditional electrical stimulation, which is a pulse signal with a low frequency (i.e., 10 KHz), it is easy to cause the tingling sensation of the user or discomfort of the user due the paresthesia. In an embodiment of the disclosure, the electrical stimulation signal is a pulse signal with a high frequency (i.e., 500 KHz). Therefore, no paresthesia, or only a very slight paresthesia, may be caused.

According to an embodiment of the disclosure, the storage device 260 may be a volatile memory (i.e., random access memory (RAM)), a non-volatile memory (i.e., flash memory), a read only memory (ROM), a hard disk or a combination thereof. The storage device 260 may be configured to store files and data required for electrical stimulation. According to an embodiment of the disclosure, the storage device 260 may be configured to store the relevant information of a look-up table provided by the external control device 200.

FIG. 4 is a schematic view of a non-implantable electrical stimulation device 100 according to an embodiment of the disclosure. As shown in FIG. 4, the electrical stimulation signal generating circuit 220 may include a variable resistor 221, a waveform generator 222, a differential amplifier 223, a channel switch circuit 224, a first resistor 225 and a second resistor 226. The measurement circuit 230 may include a current measurement circuit 231 and a voltage measurement circuit 232. It should be noted that the schematic view shown in FIG. 4 is only for the convenience of explaining the embodiment of the disclosure, but the disclosure is not limited to FIG. 4. The non-implantable electrical stimulation device 100 may also include other components, or include other equivalent circuits.

As shown in FIG. 4, according to an embodiment of the disclosure, the variable resistor 221 may be coupled to a serial peripheral interface (SPI) (not shown) of the control unit 240. The control unit 240 may transmit the command to the variable resistor 221 through the serial peripheral interface to adjust the resistance value of the variable resistor 221, so as to adjust the magnitude of the electrical stimulation signal to be output. The waveform generator 222 may be coupled to a pulse width modulation (PWM) signal generator (not shown) of the control unit 240. The pulse width modulation signal generator may generate a square wave signal and transmit the square wave signal to the waveform generator 222. After the waveform generator 222 receives the square wave signal generated by the pulse width modulation signal generator, the waveform generator 222 may convert the square wave signal into a sine wave signal, and transmit the sine wave signal to the differential amplifier 223. The differential amplifier 223 may convert the sine wave signal into a differential signal (i.e., the output electrical stimulation signal), and transmit the differential signal to the channel switch circuit 224 through the first resistor 225 and the second resistor 226. The channel switch circuit 224 may sequentially transmit the differential signal (i.e., the output electrical stimulation signal) to the electrodes corresponding to each channel according to the instruction of the control unit 240.

As shown in FIG. 4, according to an embodiment of the disclosure, the current measurement circuit 231 and the voltage measurement circuit 232 may be coupled to the differential amplifier 223, so as to obtain the current value and the voltage value of the differential signal (i.e., the output electrical stimulation signal). In addition, the current measurement circuit 231 and the voltage measurement circuit 232 may be configured to measure the voltage value and the current value of the tissue of the target area of the living body (i.e., the body of the user or patient). Furthermore, the current measurement circuit 231 and the voltage measurement 232 may be coupled to an input/output (I/O) interface (not shown) of the control unit 240, so as to receive the instruction from the control unit 240. According to the instruction of the control unit 240, the current measurement circuit 231 and the voltage measurement 232 may adjust the current and the voltage of the electrical stimulation signal to the current value and the voltage value suitable for processing by the control unit 240. For example, if the voltage value measured by the voltage measurement circuit 232 is ±10V and the voltage value suitable for processing by the control unit 240 is 0˜3V, the voltage measurement circuit 232 may firstly reduce the voltage value to ±1.5V and then raise the voltage value to 0˜3V according to the instruction of the control unit 240.

After the current measurement circuit 231 and the voltage measurement circuit 232 adjust the current value and the voltage value, the current measurement circuit 231 and the voltage measurement circuit 232 may transmit the adjusted electrical stimulation signal to an analog-to-digital convertor (ADC) (not shown) of the control unit 240. The analog-to-digital convertor may sample the electrical stimulation signal to provide the control unit 240 for subsequent calculation and analysis.

According to an embodiment of the disclosure, when the electrical stimulation is to be performed on a target area of the body of a patient, the user (which may be a medical staff or the patient himself) may select an electrical stimulation lever from a plurality of electrical stimulation levels on the operation interface of the external control device 200. In an embodiment of the disclosure, different electrical stimulation levels may correspond to different target energy values. The target energy value may be a set of preset energy values. When the user selects an electrical stimulation level, the non-implantable electrical stimulation device 100 may know how many millijoules of energy to provide to the target area for electrical stimulation according to the target energy value corresponding to the electrical stimulation level selected by the doctor or the user. According to an embodiment of the disclosure, during a trial phase, the plurality of target energy values corresponding to the plurality of electrical stimulation levels may be regarded as a first group of preset target energy values. According to an embodiment of the disclosure, the first group of preset target energy values (i.e., the plurality of target energy values) may be a linear sequence, an arithmetic sequence, or a proportional sequence, but the disclosure is not limited thereto.

According to an embodiment of the disclosure, before the non-implantable electrical stimulation device 100 performs electrical stimulation of the target area, the control unit 240 of the non-implantable electrical stimulation device may determine whether the signal quality of the electrical stimulation signal generated by the electrical stimulation signal generating circuit 220 conforms to a threshold value standard. There will be a more detailed description below.

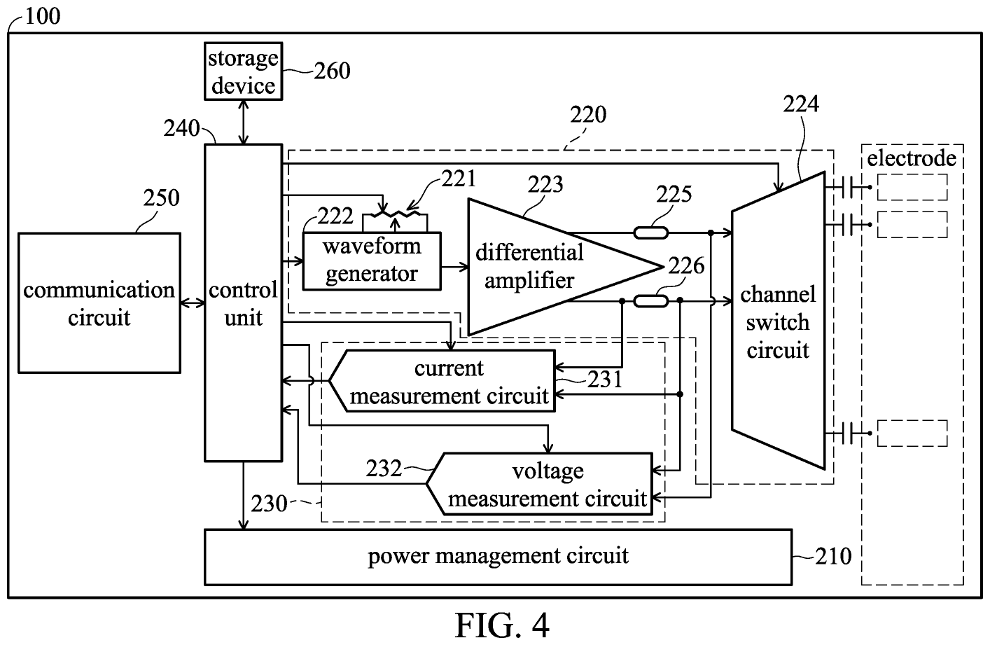

FIG. 5 is block diagram of a control unit 240 according to an embodiment of the disclosure. As shown in FIG. 5, the control unit 240 may include a sampling module 241, a fast Fourier conversion operation module 242, a determination module 243 and a calculation module 244. It should be noted that the block diagram shown in FIG. 5 is only for the convenience of explaining the embodiment of the disclosure, but the disclosure is not limited to FIG. 5. The control unit 240 may also include other components. In an embodiment of the disclosure, the sampling module 241, the fast Fourier conversion operation module 242, the determination module 243 and the calculation module 244 may be implemented by hardware or software. Furthermore, according to another embodiment of the disclosure, the sampling module 241, the fast Fourier conversion operation module 242, the determination module 243 and the calculation module 244 may also independent of the control unit 240.

According to an embodiment of the disclosure, when the control unit 240 of the non-implantable electrical stimulation device 100 determines whether the signal quality of the electrical stimulation signal generated by the electrical stimulation signal generating circuit 220 conforms to the threshold value standard, the sampling module 241 may firstly sample the electrical stimulation signal generated by the electrical stimulation signal generating circuit 220 and transmit the electrical stimulation signal to the fast Fourier conversion operation module 242 to perform a fast Fourier conversion operation. More specifically, the sampling module 241 may sample the voltage signal of the electrical stimulation signal, and the fast Fourier conversion operation module 242 may perform the fast Fourier conversion operation on the sampled voltage signal. Furthermore, the sampling module 241 may sample the current signal of the electrical stimulation signal, and the fast Fourier conversion operation module 242 may perform the fast Fourier conversion operation on the sampled current signal. In an embodiment of the disclosure, the sampling module 241 samples the electrical stimulation signal in a sampling period, and the sampling period represents sampling the voltage signal and the current signal for a period of time in the pulses included in each duration time T_{d}, i.e., sampling the electrical stimulation signal represents sampling the pulse signal. According to an embodiment of the disclosure, the sampling module 241 firstly samples the voltage signal of the electrical stimulation signal (for example, taking 512 points), and then samples the current signal of the electrical stimulation signal (for example, taking 512 points), but the disclosure is not limited to the sampling number or the sampling order.

In an embodiment of the disclosure, the sampling module 241 samples each of the pulse signals in a plurality of pulse signals. In another embodiment of the disclosure, the sampling module 241 samples at least one of the plurality of pulse signals. For example, in every two pulse signals, the sampling module 241 samples only one pulse signal, or in every three pulse signals, the sampling module 241 samples only one pulse signal. In an embodiment of the disclosure, for an unsampled pulse signal, the data of the adjacent sampled signal may be applied, but the disclosure is not limited thereto. In other words, in an embodiment of the disclosure, in a course of electrical stimulation (i.e., completing the transmission of the first target energy value or the second energy value to the target area), the sampling module 241 may sample at least one of the plurality of pulse signals once or multiple times to correspondingly obtain a tissue impedance value or a plurality of tissue impedance values.

The determination module 243 may determine whether the signal quality of the electrical stimulation signal through the fast Fourier conversion operation conforms to the threshold value standard. More specifically, the determination module 243 may determine whether a first frequency of the voltage signal through the fast Fourier conversion operation and a second frequency of the current signal through the fast Fourier conversion operation conform to a predetermined frequency, so as to determine whether the signal quality of the electrical stimulation signal conforms to the threshold value standard. That is, when the first frequency of the voltage signal through the fast Fourier conversion operation and the second frequency of the current signal through the fast Fourier conversion operation conform to the predetermined frequency, the determination module 243 may determine that the signal quality of the electrical stimulation signal conforms to the threshold value standard. When the first frequency of the voltage signal through the fast Fourier conversion operation and the second frequency of the current signal through the fast Fourier conversion operation do not conform to the predetermined frequency, the determination module 243 may determine that the signal quality of the electrical stimulation signal does not conform to the threshold value standard. According to an embodiment of the disclosure, the predetermined frequency may be between 1 KHz and 1 MHz. According to another embodiment of the disclosure, the predetermined frequency may be between 480 KHz and 520 KHz.

According to an embodiment of the disclosure, the non-electrical stimulation phase refers to in a situation in which the electrical stimulation system 100 and the external control device 200 are just powered on and connected, or after the electrical stimulation device 100 and the external control device 200 are connected, the user has not started the synchronization process of electrical stimulation, or the electrical stimulation device 100 has been attached to the skin of the user and powered on, but the course of providing electrical stimulation has not yet started. The electrical stimulation phase refers to a situation in which the electrical stimulation device 100 has started to provide the course of electrical stimulation. In the non-electrical stimulation phase, when at least one of the first frequency and the second frequency does not conform to the predetermined frequency, the determination module 243 may determine whether the voltage value corresponding to the electrical stimulation signal is greater than or equal to a predetermined voltage value (such as 2 volts). If the voltage value is less than the predetermined voltage value, the determination module 243 may increase the voltage value of the electrical stimulation signal by a preset value, and sample the electrical stimulation signal again. If the voltage value is greater than or equal to the predetermined voltage value, the determination module 243 may report the external control device 200 that the tissue impedance value may not calculated. According to an embodiment of the disclosure, the preset value may be a certain value between 0.1 and 0.4 volts, and the predetermined voltage value may be a certain value between 1 and 4 volts, but the disclosure is not limited thereto. According to an embodiment of the disclosure, an initial voltage value of the electrical stimulation signal is also a certain value between 0.1 and 0.4 volts. In the embodiment, when the first frequency or the second frequency does not conforms to the predetermined frequency, the determination module 243 may also increase a value of a counter by one, and determine whether the value of the counter is equal to a predetermined count value. When the value of the counter is equal to the predetermined count value, the determination module 243 may report the external control device 200 that the tissue impedance value may not calculated. When the value of the counter is less than the predetermined count value, the determination module 243 may determine whether the voltage value corresponding to the electrical stimulation signal is greater than or equal to a predetermined voltage value. Before the value of the counter reaches the predetermined count value, when the first frequency and the second frequency conform to the predetermined frequency once, the counter returns to zero. According to an embodiment of the disclosure, the predetermined count value may be any value between 10 and 30 times.

According to an embodiment of the disclosure, in the non-electrical stimulation phase, when at least one of the first frequency and the second frequency does not conform to the predetermined frequency, the determination module 243 may determine whether the average current value corresponding to the electrical stimulation signal is greater than or equal to a predetermined current value (such as 2 mA). If the average current value is less than the predetermined current value, the determination module 243 may increase the voltage value of the electrical stimulation signal by a preset value. If the average current value is greater than or equal to the predetermined current value, the determination module 243 may perform the subsequent operation of the electrical stimulation signal. According to an embodiment of the disclosure, the preset value may be a certain value between 0.1 and 0.4 volts, and the predetermined voltage value may be a certain value between 1 and 4 volts, but the disclosure is not limited thereto. According to an embodiment of the disclosure, an initial voltage value of the electrical stimulation signal is also a certain value between 0.1 and 0.4 volts.

According to an embodiment of the disclosure, in the electrical stimulation phase, when at least one of the first frequency and the second frequency does not conform to the predetermined frequency, the determination module 243 may sample the electrical stimulation signal, and does not use the electrical stimulation signal sampled this time, or the external control device 200 may know not to use the electrical stimulation signal sampled this time according to the determination result of the determination module 243. In the embodiment, when at least one of the first frequency and the second frequency does not conform to the predetermined frequency, the determination module 243 may use the previous electrical stimulation signal conforming to the threshold value standard for subsequent operation of electrical stimulation, or the external control device 200 may use the previous electrical stimulation signal conforming to the threshold value standard for subsequent operation of electrical stimulation according to the determination result of the determination module 243.

According to an embodiment of the disclosure, when the determination module 243 determines that the signal quality of the electrical stimulation signal conforms to the threshold value standard, the calculation module 244 may calculate the impedance value (i.e., the tissue impedance value) corresponding to the sampled electrical stimulation signal, so as to perform electrical stimulation of a target area. There will be a more detailed description below.

According to an embodiment of the disclosure, when the determination module 243 determines that the signal quality of the electrical stimulation signal conforms to the threshold value standard, the calculation module 244 may extract a first voltage sampling point corresponding to the maximum voltage value and a second voltage sampling point the minimum voltage value in each sampling period, and subtract the maximum voltage value and the minimum voltage value and divide them by 2 to generate an average voltage value, thereby eliminating the background value. It should be noted that, as mentioned above, the voltage measurement circuit 232 may raise the voltage value to a positive value according to the command of the control unit 240 for the control unit 240 to process. Furthermore, when the determination module 243 determines that the signal quality of the electrical stimulation signal conforms to the threshold value standard, the calculation module 244 may extract a first current sampling point corresponding to the maximum current value and a second current sampling point corresponding to the minimum current value in each sampling period, and subtract the maximum current value and the minimum value and divide them by 2 to generate the average current value and eliminate the background value. After obtaining the average voltage value and the average current value, the calculation module 244 may obtain the total impedance value according to the average voltage value and the average current value, and calculate the tissue impedance value according to the total impedance value. Below there will be a more detailed description of how to calculate the tissue impedance value according to the total impedance value. According to another embodiment of the disclosure, if the background value is 0, the calculation module 244 may add the maximum voltage value and the minimum voltage value and divide them by 2 to generate the average voltage value, and add the maximum current value and the minimum current value and divide them by 2 to generate the average current value.

According to another embodiment of the disclosure, when the determination module 243 determines that the signal quality of the electrical stimulation signal conforms to the threshold value standard, the sampling module 241 may sample all the peaks and valleys of the voltage signal of the electrical stimulation signal, and the calculation module 244 may generate an average voltage value according to the values of all the voltage sampling points. For example, the calculation module 244 may average the peaks and valleys included in 512 sampling points of the voltage signal obtained in each sampling period to generate an average voltage value. Furthermore, the sampling module 241 may sample all the peaks and valleys of the current signal of the electrical stimulation signal, and the calculation module 244 may generate the average current value according to the values of all the current sampling points. For example, the calculation module 244 may average the peaks and valleys included in 512 sampling points of the current signal obtained in each sampling period to generate the average current value. Then, the calculation module 244 may obtains the total impedance value according to the average voltage value and the average current value, and calculate the tissue impedance value according to the total impedance value. Below there will be a more detailed description of how to calculate the tissue impedance value according to the total impedance value.

According to an embodiment of the disclosure, before the non-implantable electrical stimulation device 100 performs the electrical stimulation on the target area, such as in the non-electrical stimulation state, the non-implantable electrical stimulation device 100 may calculate the tissue impedance value of the target area, and the obtained tissue impedance value may then be used to calculate the energy value of the electrical stimulation signal transmitted to the target area. According to an embodiment of the disclosure, such as non-implantable electrical stimulation device 100 shown in FIG. 1A, FIG. 1B and FIG. 1C, the non-implantable electrical stimulation device 100 may calculate the tissue impedance value according to an impedance value of the electrode assembly 120 and an impedance of the electrical stimulator 110. There will be a more detailed description below.

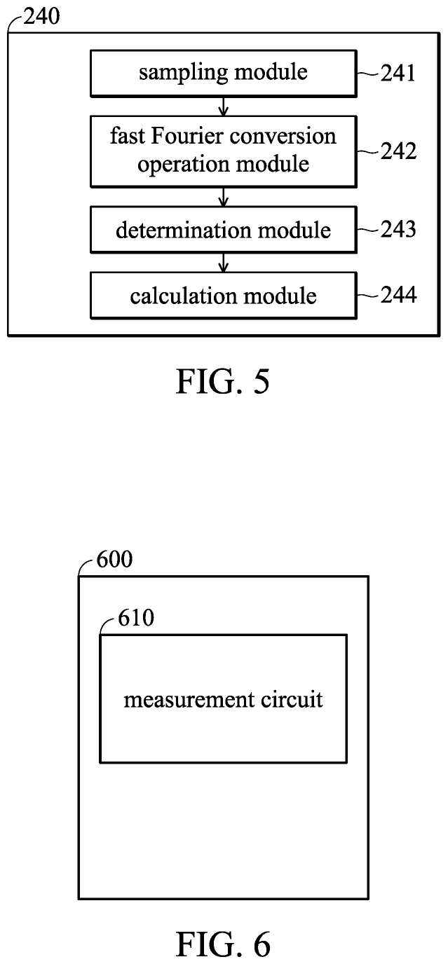

FIG. 6 is block diagram of an impedance compensation device 600 according to an embodiment of the disclosure. As shown in FIG. 6, the impedance compensation device 500 may include a measurement circuit 610, but the disclosure is not limited thereto. The measurement circuit 610 may be configured to measure the impedance value Z_{Inner} of the electrical stimulator 110 and the impedance value Z_{Electrode} of the electrode assembly 120. According to an embodiment of the disclosure, the impedance compensation device 600 (or the measurement circuit 610) may also include the related circuit structure shown in FIG. 4.

According to an embodiment of the disclosure, when the measurement circuit 610 is to measure the non-implantable electrical stimulation device 100 shown in FIG. 1A, FIG. 1B and FIG. 1C, the measurement circuit 610 may provide a high-frequency environment. The frequency is the same as the frequency of the electrical stimulation signal for electrical stimulation of the target area. Herein, the frequency is taken 500 kHz as an example. Then, the measurement circuit 610 may measure the resistance value R_{Electrode}, the capacitance value C_{Electrode} and the inductance value L_{Electrode} of the electrode assembly 120, and calculate the impedance value Z_{Electrode} of the electrode assembly 120 under the high-frequency signal according to at least one of the measured resistance value R_{Electrode}, capacitance value C_{Electrode}, and inductance value L_{Electrode}. Furthermore, the measurement circuit 610 may measure the resistance value R_{Inner}, the capacitance value C_{Inner} and the inductance value L_{Inner} of the electrical stimulator 110, and calculate the impedance value Z_{Inner} of the electrical stimulator 110 according to at least one of the measured resistance value R_{Inner}, capacitance value C_{Inner}, and inductance value L_{Inner}. In an embodiment of the disclosure, the inductance value L_{Inner} of the electrical stimulator 110 may not be measured. The measurement circuit 610 may write the calculated impedance value Z_{Electrode} of the electrode assembly 120 and the calculated impedance value Z_{Inner} of the electrical stimulator 110 into the firmware of the non-implantable electrical stimulation device 100. It should be noted that the impedance value Z_{Electrode} of the electrode assembly 120 is the overall impedance value of the body 121, the two electrodes 122, the at least one second magnetic unit 123, the at least two electrical connectors 124, and the conductive gel 125.

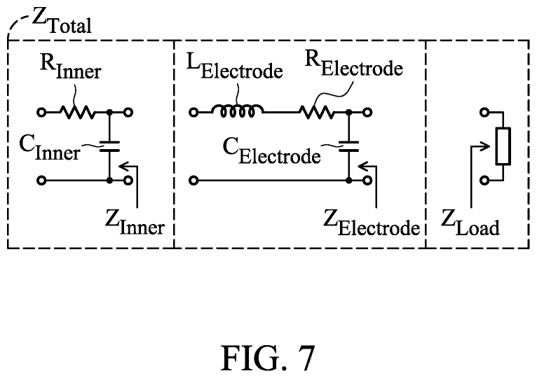

When the non-implantable electrical stimulation device 100 is to calculate the tissue impedance value Z_{Load} of the target area, the non-implantable electrical stimulation device 100 may deduct the impedance value Z_{Electrode} of the electrode assembly 120 and the impedance value Z_{Inner} of the electrical stimulator 110 from the measured total impedance value Z_{Total}, so as to obtain the tissue impedance value Z_{Load} of the target area, such as the impedance compensation model shown in FIG. 7, Z_{Load}=Z_{Total}−Z_{Inner}−Z_{Electrode}, but the disclosure is not limited thereto. In an embodiment of the disclosure, the total impedance value Z_{Total} may be calculated by the calculation module 244 according to the current measured by the current measurement circuit 231 and the voltage measured by the voltage measurement circuit 232 (i.e., R=V/I). Since the calculation manner of the impedance value Z_{Electrode} of the electrode assembly 120 and the impedance value Z_{Inner} of the electrical stimulator 110 may refer to Z=R+j (XL−XC), wherein R is the resistance, XL is the inductive reactance and XC is the capacitive reactance, Z=R+j (XL−XC) is well known to those skilled in the art, and the description thereof is not repeated herein.

According to an embodiment of the disclosure, the measurement circuit 610 may simulate a high-frequency environment according to an electrical stimulation frequency used by the non-implantable electrical stimulation device 100. According to an embodiment of the disclosure, the pulse frequency range of the high-frequency environment provided by the measurement circuit 610 may be in the range of 1 KHz to 1000 KHz. According to an embodiment of the disclosure, the pulse frequency of the high-frequency environment provided by the measurement circuit 610 is the same as that of the electrical stimulation signal.

According to an embodiment of the disclosure, the impedance compensation device 500 may be configured in the external control device 200. According to another embodiment of the disclosure, the impedance compensation device 500 may be configured in the non-implantable electrical stimulation device 100. That is, the high-frequency environment may be provided by the non-implantable electrical stimulation device 100 or the external control device 200. Furthermore, according to another embodiment of the disclosure, the impedance compensation device 500 may also be a stand-alone device (i.e., an impedance analyzer).

According to an embodiment of the disclosure, the impedance compensation device 500 may be applied before the non-implantable electrical stimulation device 100 is produced (i.e., in the laboratory or factory). In an embodiment, before the non-implantable electrical stimulation device 100 is produced, the impedance compensation device 600 may firstly calculate the impedance value Z_{Electrode} of the electrode assembly 120 and the impedance value Z_{Inner} of the electrical stimulator 110, and then write the calculated impedance value Z_{Electrode} of the electrode assembly 120 and the calculated impedance value Z_{Inner} of the electrical stimulator 110 into the firmware of the non-implantable electrical stimulation device 100. According to an embodiment of the disclosure, in the electrical stimulation phase and the non-electrical stimulation phase, the impedance compensation device 600 may also perform real-time compensation, i.e., Z_{Inner} and Z_{Electrode} may be measured and obtained every time an electrical stimulation signal is sent.

According to an embodiment of the disclosure, after the non-implantable electrical stimulation device 100 obtains the tissue impedance value Z_{Load}, the non-implantable electrical stimulation device 100 may transmit the tissue impedance value Z_{Load} to the external control device 200. The external control device 200 may determine whether the tissue impedance value Z_{Load} is within a predetermined range. In the electrical stimulation phase, when the tissue impedance value Z_{Load} is outside the predetermined range, the external control device 200 may instruct the electrical stimulator 110 (the non-implantable electrical stimulation device 100) to stop the electrical stimulation. In the electrical stimulation phase, when the tissue impedance value Z_{Load} is within the predetermined range, the external control device 200 may instruct the electrical stimulator 110 (the non-implantable electrical stimulation device 100) to continue the electrical stimulation. According to another embodiment of the disclosure, the non-implantable electrical stimulation device 100 may also determine by itself whether the tissue impedance value Z_{Load} is within a predetermined range. In the electrical stimulation phase, when the tissue impedance value Z_{Load} is outside the predetermined range, the non-implantable electrical stimulation device 100 (or the electrical stimulator 110) may stop the electrical stimulation. In the electrical stimulation phase, when the tissue impedance value Z_{Load} is within the predetermined range, the non-implantable electrical stimulation device 100 (or the electrical stimulator 110) may continue the electrical stimulation. According to an embodiment of the disclosure, when the tissue impedance value is outside the predetermined range, it indicates that the electrical stimulator 110 (the non-implantable electrical stimulation device 100) and the electrode assembly 120 are in an open circuit. When the tissue impedance value is within the predetermined range, it indicates that the electrical stimulator 110 and the electrode assembly 120 are normally electrically connected.

According to an embodiment of the disclosure, the upper limit value of the predetermined range of the tissue impedance may be 2000 ohms, and the lower limit value of the predetermined range of the tissue impedance may be 70 ohms.

According to an embodiment of the disclosure, after the non-implantable electrical stimulation device 100 obtains a plurality of tissue impedance values Z_{Load} (such as three tissue impedance values Z_{Load}), the calculation module 244 may calculate the tissue impedance average value of the plurality of tissue impedance values, and transmit the tissue impedance average value to the external control device 200. According to an embodiment of the disclosure, the non-implantable electrical stimulation device 100 may determine whether the tissue impedance average value is greater than the previous tissue impedance average value, whether the difference (i.e. absolute difference) between the tissue impedance average value and the previous tissue impedance average value is greater than a first predetermined ratio (such as 3%, 5% or 10%). When the tissue impedance average value is greater than the previous tissue impedance average value and the difference between the tissue impedance average value and the previous tissue impedance average value is greater than the first predetermined ratio, the non-implantable electrical stimulation device 100 may average the tissue impedance average value and the previous tissue impedance average value to generate an average value, and update an output tissue impedance average value according to the average value. When he tissue impedance average value is not greater than the previous tissue impedance average value or the difference between the tissue impedance average value and the previous tissue impedance average value is not greater than first predetermined ratio, the non-implantable electrical stimulation device 100 updates the tissue impedance average value to the output tissue impedance average value.

Furthermore, according to an embodiment of the disclosure, the non-implantable electrical stimulation device 100 determine whether the absolute value of the difference between the output tissue impedance average value and the previous output impedance average value is greater than a second predetermined ratio (such as 3%, 5% or 10%). When the difference between the output tissue impedance average value and the previous output impedance average value is not greater than the second predetermined ratio, the external control device 200 instructs the electrical stimulator 110 (the non-implantable electrical stimulation device 100) to not adjust an output current, wherein the output current refers to a current of the electrical stimulation signal generated by the non-implantable electrical stimulation device 100. It should be noted that different output tissue impedance average values correspond to different output current. When the output tissue impedance average value is higher, the output current is also higher. In an embodiment of the disclosure, the corresponding relationship between the output tissue impedance average value and the output current may be stored in a look-up table (not shown). When the difference between the output tissue impedance average value and the previous output tissue impedance average value is greater than the second predetermined ratio, the non-implantable electrical stimulation device 100 determines whether the output tissue impedance average value is less than a predetermined impedance value (such as 2000 ohms). When the output tissue impedance average value is not less than (i.e., greater than or equal to) the predetermined impedance value, the non-implantable electrical stimulation device 100 instructs the electrical stimulator 110 to not adjust the output current. When the output tissue impedance average value is less than the predetermined impedance value, the non-implantable electrical stimulation device 100 adjusts the output current according to the tissue impedance average value.

For example, when the tissue impedance values obtained by the non-implantable electrical stimulation device 100 for the first to three times are 290, 300, and 310 ohms, the tissue impedance average value is 300 ohms. When the tissue impedance values obtained by the non-implantable electrical stimulation device 100 for the fourth 4 to sixth times are 270, 280, and 290 ohms, the (new) tissue impedance average value is 280 ohms. At this time, the tissue impedance average value (280 ohms) is less than the previous tissue impedance average value (300 ohms), and the non-implantable electrical stimulation device 100 updates 280 ohms to the output tissue impedance average value. When the tissue impedance values obtained by the non-implantable electrical stimulation device 100 for the seventh to ninth times are 340, 350, and 360 ohms, the tissue impedance average value is 350 ohms. At this time, the tissue impedance average value (350 ohms) is greater than the previous tissue impedance average value (280 ohms), and the absolute value of the difference is greater than the first predetermined ratio (such as 10%), the non-implantable electrical stimulation device 100 averages the current tissue impedance average value (350 ohms) and the previous tissue impedance average value (280 ohms) to generate an average value (315 ohms), and updates the output tissue impedance average value according to the average value. Then, the non-implantable electrical stimulation device 100 determines that the absolute value of the difference between the output tissue impedance average value (315 ohms) and the previous tissue impedance average value (280 ohms) is greater than the second predetermined ratio (such as 5%), the non-implantable electrical stimulation device 100 determines that the output tissue impedance average value (315 ohms) is less than the predetermined impedance value (such as 2000 ohms), and the non-implantable electrical stimulation device 100 adjusts the output current according to the current output tissue impedance average value (315 ohms).

In an embodiment of the disclosure, the tissue impedance, the tissue impedance average value and the output tissue impedance average value obtained each time may be stored in a buffer area of the control unit 240 or a buffer area of the storage device 260, but the disclosure is not limited thereto.

According to an embodiment of the disclosure, in the electrical stimulation phase (i.e., when the non-implantable electrical stimulation device 100 has provided the treatment of the electrical stimulation), in order to make the measurement circuit 130 to operate smoothly, if the voltage of the electrical stimulation signal is greater than a predetermined voltage value (such as 7.5 volts), the non-implantable electrical stimulation device 100 generates a first predetermined number (such as 13) of electrical stimulation signals, performs a buck operation on a second predetermined number of electrical stimulation signals in the first predetermined number of electrical stimulation signals (i.e., the voltage is bucked to the predetermined voltage value), and use the second predetermined number of electrical stimulation signals through the buck operation to calculate the subsequent tissue impedance value. The non-bucked electrical stimulation signal may not be used to calculate the subsequent tissue impedance value. The non-implantable electrical stimulation device 100 may repeat this manner. That is, after generating the number first predetermined number of electrical stimulation signals, the second predetermined number of electrical stimulation signals are generated and bucked to the predetermined voltage value, and then the first predetermined number of electrical stimulation signals are generated. For example, in the electrical stimulation phase, if the voltages of previous N times (such as N=10, i.e., first to tenth times) of the first predetermined number (such as 13) of electrical stimulation signals are greater than the predetermined voltage value (such as 7.5 volts), the N times of electrical stimulation signals may not be used to calculate the subsequent tissue impedance value, and the non-implantable electrical stimulation device 100 may only preform the buck operation (for example, bucking to 7.5 volts) on the second predetermined number of electrical stimulation signals (such as eleventh to thirteen times), and use the bucked specific electrical stimulation signals to calculate the subsequent tissue impedance value.

In an embodiment of the disclosure, the tissue impedance value is used to calculate the energy value of the electrical stimulation signal transmitted to the target area, and the calculation manner of the energy value of the electrical stimulation signal may be E=0.5*I²*Z_{Load}*PW*rate*t, wherein E is the energy value, in joules, and 0.5 is a constant; I is the current, in amperes; PW is duration time T_{d} of the pulse, in seconds; Z_{Load} is the tissue impedance value, in ohms; rate is the pulse repetition frequency of the electrical stimulation signal, in Hz; t is a time for electrical stimulation, in seconds.

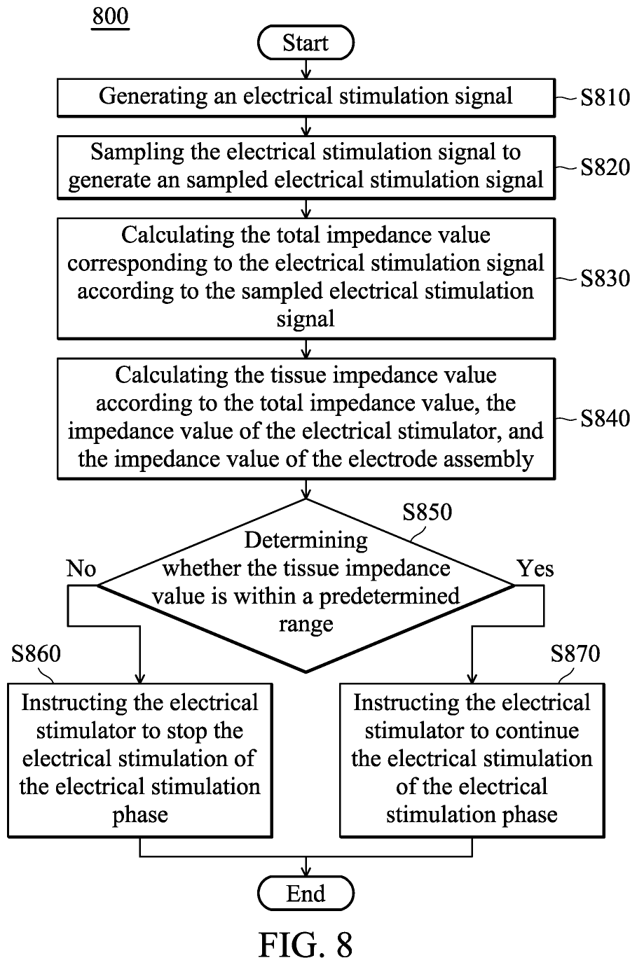

FIG. 8 is a flowchart 800 of an impedance monitoring method according to an embodiment of the disclosure. The flowchart 800 of the impedance monitoring method is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. The electrical stimulator 110 may store the impedance value of the electrical stimulator 110 and the impedance value of the electrode assembly 120, and the impedance value of the electrical stimulator 110 and the impedance value of the electrode assembly 120 is obtained at the same frequency of the electrical stimulation signal. As shown in FIG. 8, in step S810, the electrical stimulator 110 (the non-implantable electrical stimulation device 100) generates an electrical stimulation signal.

In step S820, the electrical stimulator 110 (the non-implantable electrical stimulation device 100) samples the electrical stimulation signal to generate an sampled electrical stimulation signal.

In step S830, the electrical stimulator 110 (the non-implantable electrical stimulation device 100) calculates the total impedance value corresponding to the electrical stimulation signal according to the sampled electrical stimulation signal.

In step S840, the electrical stimulator 110 (the non-implantable electrical stimulation device 100) calculates the tissue impedance value according to the total impedance value, the impedance value of the electrical stimulator 110, and the impedance value of the electrode assembly 120.

In step S850, the external control device 200 receives the tissue impedance value from the electrical stimulator 110 (the non-implantable electrical stimulation device 100) and determines whether the tissue impedance value is within a predetermined range.

In another embodiment of the disclosure, the electrical stimulator 110 (the non-implantable electrical stimulation device 100) may also determine by itself whether the tissue impedance value is within a predetermined range.

In an electrical stimulation phase, when the tissue impedance value is outside the predetermined range, step S860 is performed. In step S860, the external control device 200 instructs the electrical stimulator 110 (the non-implantable electrical stimulation device 100) to stop the electrical stimulation of the electrical stimulation phase.

In another embodiment of the disclosure, the electrical stimulator 110 (the non-implantable electrical stimulation device 100) may also stop the electrical stimulation of the electrical stimulation phase by itself.

In the electrical stimulation phase, when the tissue impedance value is within the predetermined range, step S870 is performed. In step S870, the external control device 200 instructs the electrical stimulator 110 (the non-implantable electrical stimulation device 100) to continue the electrical stimulation of the electrical stimulation phase.

In another embodiment of the disclosure, the electrical stimulator 110 (the non-implantable electrical stimulation device 100) may also continue the electrical stimulation of the electrical stimulation phase by itself.

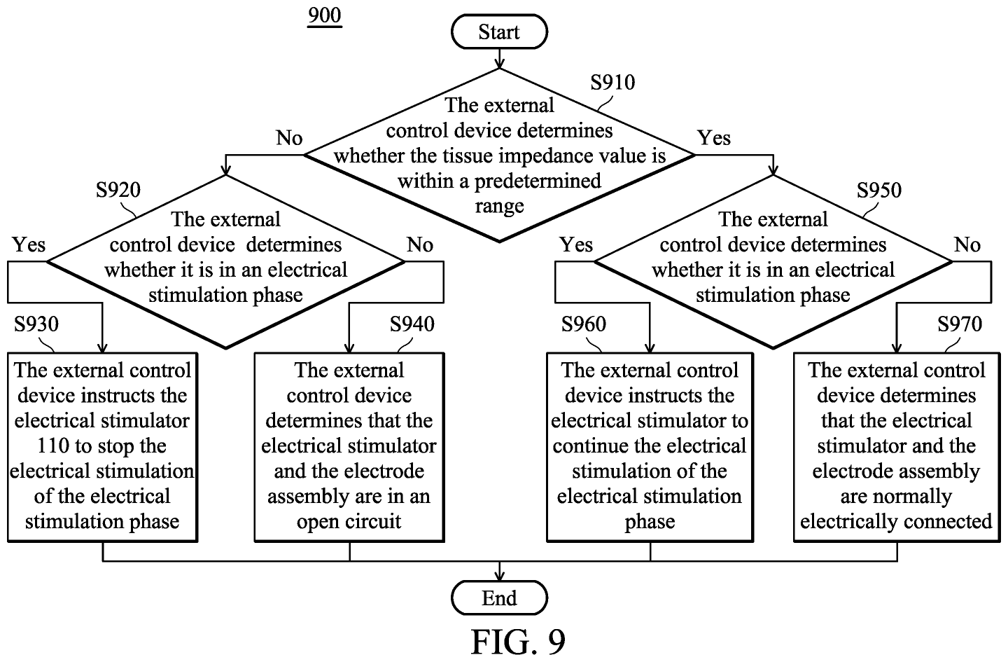

FIG. 9 is a flowchart 900 of an impedance monitoring method according to another embodiment of the disclosure. The flowchart 900 of the impedance monitoring method is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. The electrical stimulator 110 may store the impedance value of the electrical stimulator 110 and the impedance value of the electrode assembly 120, and the impedance value of the electrical stimulator 110 and the impedance value of the electrode assembly 120 is obtained at the same frequency of the electrical stimulation signal. As shown in FIG. 9, in step S910, the external control device 200 determines whether the tissue impedance value is within a predetermined range.

When the tissue impedance value is outside the predetermined range, step S920 is performed. In step S920, the external control device 200 determines whether it is in an electrical stimulation phase. When it is in the electrical stimulation phase, step S930 is performed. In step S930, the external control device 200 instructs the electrical stimulator 110 (the non-implantable electrical stimulation device 100) to stop the electrical stimulation of the electrical stimulation phase. When it is in a non-electrical stimulation phase, step S940 is performed. In step S940, the external control device 200 determines that the electrical stimulator 110 (the non-implantable electrical stimulation device 100) and the electrode assembly 120 are in an open circuit.

When the tissue impedance value is within the predetermined range, step S950 is performed. In step S950, the external control device 200 determines whether it is in an electrical stimulation phase. When it is in the electrical stimulation phase, step S960 is performed. In step S960, the external control device 200 instructs the electrical stimulator 110 (the non-implantable electrical stimulation device 100) to continue the electrical stimulation of the electrical stimulation phase. When it is in a non-electrical stimulation phase, step S970 is performed. In step S970, the external control device 200 determines that the electrical stimulator 110 and the electrode assembly 120 are normally electrically connected.

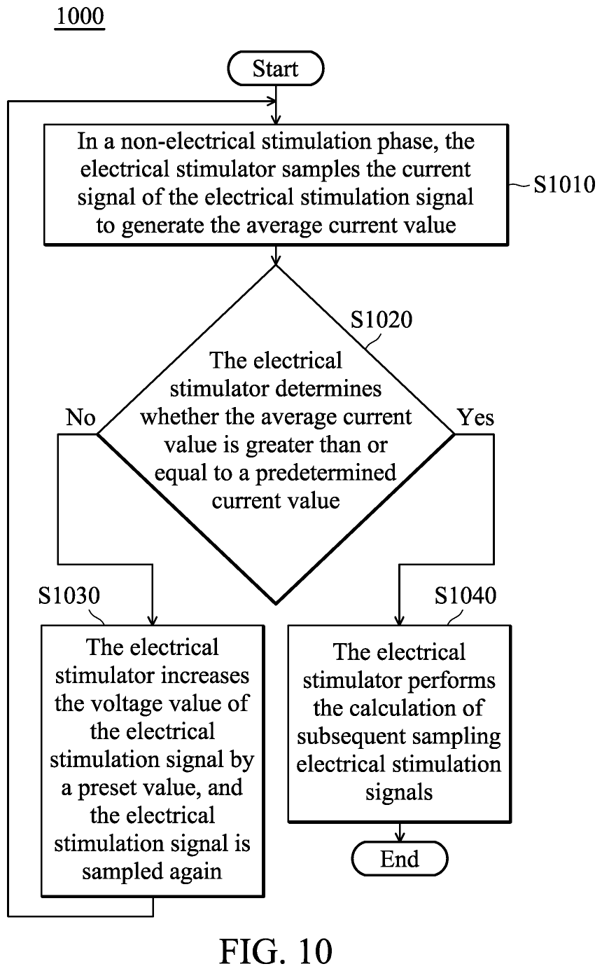

FIG. 10 is a flowchart 1000 of a processing method of an electrical stimulation signal according to an embodiment of the disclosure. The flowchart 1000 of the processing method of the electrical stimulation signal is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. As shown in FIG. 10, in step S1010, in a non-electrical stimulation phase, the electrical stimulator 110 (the non-implantable electrical stimulation device 100) samples the current signal of the electrical stimulation signal to generate the average current value.

In step S1020, the electrical stimulator 110 (the non-implantable electrical stimulation device 100) determines whether the average current value is greater than or equal to a predetermined current value.

When the average current value is less than the predetermined current value, step S1030 is performed. In step S1030, the electrical stimulator 110 (the non-implantable electrical stimulation device 100) increases the voltage value of the electrical stimulation signal by a preset value, and the electrical stimulation signal is sampled again.

When the average current value is greater than or equal to the predetermined current value, step S1040 is performed. In step S1040, the electrical stimulator 110 (the non-implantable electrical stimulation device 100) performs the calculation of subsequent sampling electrical stimulation signals.

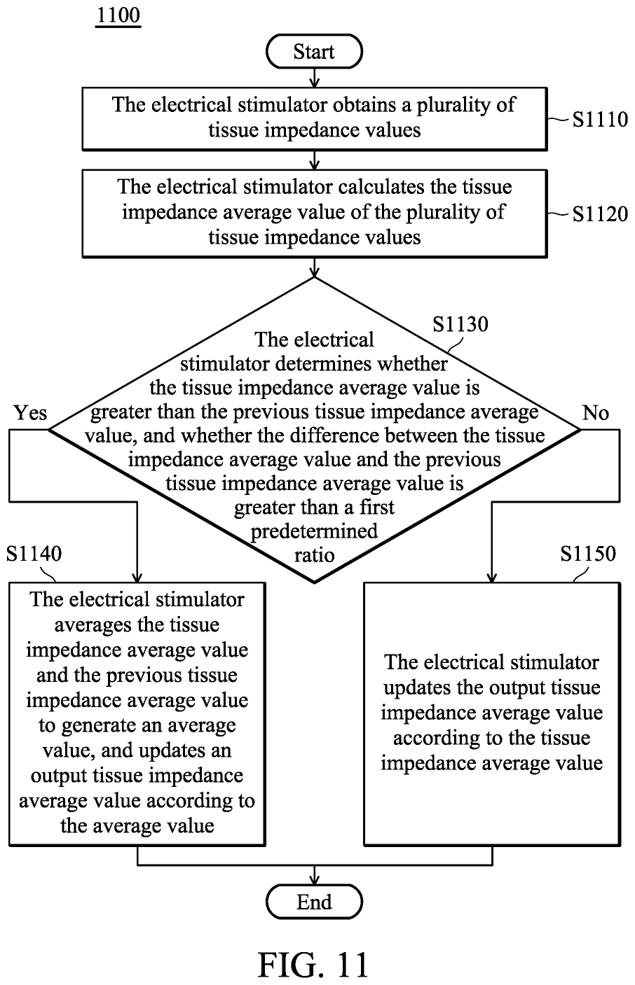

FIG. 11 is a flowchart 1110 of an updating method of an output tissue impedance average value according to an embodiment of the disclosure. The a flowchart 1110 of the updating method of the output tissue impedance average value is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. As shown in FIG. 11, in step S1110, the electrical stimulator 110 (the non-implantable electrical stimulation device 100) obtains a plurality of tissue impedance values.

In step S1120, the electrical stimulator 110 (the non-implantable electrical stimulation device 100) calculates the tissue impedance average value of the plurality of tissue impedance values.

In step S1130, the electrical stimulator 110 (the non-implantable electrical stimulation device 100) determines whether the tissue impedance average value is greater than the previous tissue impedance average value, and whether the difference between the tissue impedance average value and the previous tissue impedance average value is greater than a first predetermined ratio.

When the tissue impedance average value is greater than the previous tissue impedance average value and the difference is greater than the first predetermined ratio, step S1140 is performed. In step S1140, the electrical stimulator 110 (the non-implantable electrical stimulation device 100) averages the tissue impedance average value and the previous tissue impedance average value to generate an average value, and updates an output tissue impedance average value according to the average value.

When the tissue impedance average value is not greater than the previous tissue impedance average value or the difference is not greater than first predetermined ratio, step S1150 is performed. In step S1150, the electrical stimulator 110 (the non-implantable electrical stimulation device 100) updates the output tissue impedance average value according to the tissue impedance average value.

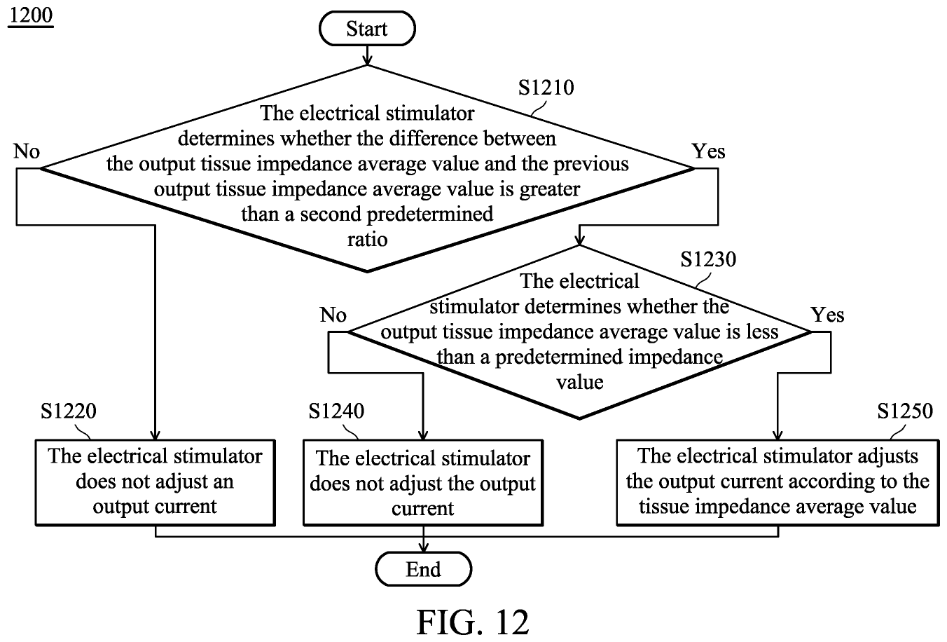

FIG. 12 is a flowchart 1200 of an adjustment method of an output current according to an embodiment of the disclosure. The flowchart 1200 of the adjustment method of the output current is applied to the non-implantable electrical stimulation device 100. The non-implantable electrical stimulation device 100 includes an electrical stimulator 110 and an electrode assembly 120. The electrical stimulator 110 is detachably electrically connected to the electrode assembly 120. As shown in FIG. 12, in step S1210, the electrical stimulator 110 (the non-implantable electrical stimulation device 100) determines whether the difference between the output tissue impedance average value and the previous output tissue impedance average value is greater than a second predetermined ratio.

When the difference is not greater than (i.e., less than or equal to) the second predetermined ratio, step S1220 is performed. In step S1220, the electrical stimulator 110 (the non-implantable electrical stimulation device 100) does not adjust an output current.

When the difference is greater than the second predetermined ratio, step S1230 is performed. In step S1230, the electrical stimulator 110 (the non-implantable electrical stimulation device 100) determines whether the output tissue impedance average value is less than a predetermined impedance value.

When the output tissue impedance average value is not less than (i.e., greater than or equal to) the predetermined impedance value, step S1240 is performed. In step S1240, the electrical stimulator 110 (the non-implantable electrical stimulation device 100) does not adjust the output current.

When the output tissue impedance average value is less than the predetermined impedance value, step S1250 is performed. In step S1250, the electrical stimulator 110 (the non-implantable electrical stimulation device 100) adjusts the output current according to the tissue impedance average value.

According to an embodiment of the disclosure, a computer-readable storage medium may store one or more instructions, and cooperate with the non-implantable electrical stimulation device 100 for providing electrical stimulation and the external control device 200. When one or more instructions stored in the computer-readable storage medium are executed by the non-implantable electrical stimulation device 100 and the external control device 200, the non-implantable electrical stimulation device 100 and the external control device 200 may perform a plurality of steps included in the impedance monitoring method.

According to the impedance monitoring method provided by the disclosure, it may determine whether the calculated tissue impedance value is within the predetermined range when performing the electrical stimulation. Therefore, it may be prevented that the calculated tissue impedance value is too large or too small to cause discomfort to the user when performing the electrical stimulation. Furthermore, traditionally, after the electrical stimulator is implanted in the human body, as time increase, the tissue impedance may change because the fact that the human tissue may coat the non-implantable electrical stimulation device and the electrode assembly, or the posture of the human body changes. Therefore, according to the impedance monitoring method provided by the disclosure, the change of the tissue impedance may be continuously monitored in a relatively real-time manner when performing the electrical stimulation.

The serial numbers in the specification and claim, such as “first”, “second”, etc., are only for the convenience of description, and there is no sequential relationship between them.

The steps of the method and the algorithm described in connection with the aspects disclosed herein may be embodied directly in hardware, in a software module executed by a processor, or in a combination of the two. A software module (e.g., including executable instructions and related data) and other data may reside in a data memory such as a random access memory RAM), a flash memory, a read-only memory (ROM), an erasable programmable read only memory (EPROM), an electrically erasable programmable read only memory (EEPROM), a register, a hard disk, a removable disk, a compact disc read only memory (CD-ROM), or any other form of computer-readable storage medium known in the art. A sample storage medium may be coupled to a machine such as, for example, a computer/processor (which may be referred to herein, for convenience, as a “processor”) such that the processor can read information (e.g., code) from and write information to the storage medium. A sample storage medium may be integral to the processor. The processor and the storage medium may reside in an application specific integrated circuit (ASIC). The ASIC may reside in user equipment. Alternatively, the processor and the storage medium may reside as discrete components in user equipment. Moreover, in some aspects any suitable computer-program product may include a computer-readable medium comprising codes relating to one or more of the aspects of the disclosure. In some aspects, a computer program product may include packaging materials.

The above paragraphs describe many aspects. Obviously, the teaching of the disclosure can be accomplished by many methods, and any specific configurations or functions in the disclosed embodiments only present a representative condition. Those who are skilled in this technology will understand that all of the disclosed aspects in the disclosure can be applied independently or be incorporated.

While the disclosure has been described by way of example and in terms of the preferred embodiments, it should be understood that the disclosure is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An impedance monitoring method, applied to a non-implantable electrical stimulation device, wherein the non-implantable electrical stimulation device comprises an electrical stimulator and an electrode assembly, the electrical stimulator is detachably electrically connected to the electrode assembly, the electrical stimulator stores an impedance value of the electrical stimulator and an impedance value of the electrode assembly, and the impedance monitoring method comprises:
using the electrical stimulator to generate an electrical stimulation signal, wherein the electrical stimulation signal performs electrical stimulation of a target area through the electrode assembly;
using the electrical stimulator to sample the electrical stimulation signal to calculate a total impedance value corresponding to the electrical stimulation signal; and
using the electrical stimulator to calculate a tissue impedance value according to the total impedance value, the impedance value of the electrical stimulator, and the impedance value of the electrode assembly, wherein the tissue impedance value is used to calculate an energy value corresponding to the electrical stimulation signal transmitted to the target area.

2. The impedance monitoring method as claimed in claim 1, wherein the electrode assembly comprises two electrodes.

3. The impedance monitoring method as claimed in claim 2, wherein the two electrodes are thin film electrodes.

4. The impedance monitoring method as claimed in claim 1, wherein the electrode assembly comprises a conductive gel.

5. The impedance monitoring method as claimed in claim 1, wherein the target area comprises a skin of a living body.

6. The impedance monitoring method as claimed in claim 1, wherein in the step of using the electrical stimulator to sample the electrical stimulation signal to calculate the total impedance value corresponding to the electrical stimulation signal, the electrical stimulation signal further comprises a plurality of pulse signals, and the electrical stimulator samples at least one of the plurality of pulse signals to calculate the total impedance value corresponding to the at least one of the plurality of pulse signals.

7. The impedance monitoring method as claimed in claim 1, further comprising:
using the electrical stimulator to stop performing electrical stimulation in the target area when the energy value generated by the electrical stimulation signal is accumulated to a target energy value.

8. The impedance monitoring method as claimed in claim 1, wherein the impedance value of the electrical stimulator and the impedance value of the electrode assembly are measured in an environment of the same frequency as the electrical stimulation signal.

9. The impedance monitoring method as claimed in claim 1, further comprising:
determining whether the tissue impedance value is within a predetermined range;
in an electrical stimulation phase, when the tissue impedance value is outside the predetermined range, instructing the electrical stimulator to stop an electrical stimulation of the electrical stimulation phase; and
in the electrical stimulation phase, when the tissue impedance value is within the predetermined range, instructing the electrical stimulator to continue the electrical stimulation of the electrical stimulation phase.

10. The impedance monitoring method as claimed in claim 1, further comprising:
in a non-electrical stimulation phase, sampling a current signal of the electrical stimulation signal to generate an average current value; and
determining whether the average current value is greater than or equal to a predetermined current value;
wherein when the average current value is less than the predetermined current value, a voltage value of the electrical stimulation signal is increased by a preset value, and the electrical stimulation signal is sampled again.

11. The impedance monitoring method as claimed in claim 1, wherein a frequency of the electrical stimulation signal is between 1 KHz and 1 MHz.

12. The impedance monitoring method as claimed in claim 9, wherein an upper limit value of the predetermined range is 2000 ohms, and a lower limit value of the predetermined range is 70 ohms.

13. The impedance monitoring method as claimed in claim 1, further comprising:
using the electrical stimulator to obtain a plurality of tissue impedance values;
using the electrical stimulator to calculate a tissue impedance average value of the plurality of tissue impedance values;
using the electrical stimulator to determine whether the tissue impedance average value is greater than a previous tissue impedance average value, and whether a difference between the tissue impedance average value and the previous tissue impedance average value is greater than a first predetermined ratio;
when the tissue impedance average value is greater than the previous tissue impedance average value and the difference is greater than the first predetermined ratio, averaging the tissue impedance average value and the previous tissue impedance average value to generate an average value, and updating an output tissue impedance average value according to the average value; and
when the tissue impedance average value is not greater than the previous tissue impedance average value or the difference is not greater than first predetermined ratio, updating the output tissue impedance average value according to the tissue impedance average value.

14. The impedance monitoring method as claimed in claim 13, further comprising:
using the electrical stimulator to determine whether a difference between the output tissue impedance average value and a previous output tissue impedance average value is greater than a second predetermined ratio;
when the difference is not greater than the second predetermined ratio, not adjusting an output current; and
when the difference is greater than the second predetermined ratio, determining whether the output tissue impedance average value is less than a predetermined impedance value;
wherein when the output tissue impedance average value is not less than the predetermined impedance value, the output current is not adjusted;
wherein when the output tissue impedance average value is less than the predetermined impedance value, the output current is adjusted according to the tissue impedance average value.

15. The impedance monitoring method as claimed in claim 1, wherein in the step of using the electrical stimulator to sample the electrical stimulation signal to calculate the total impedance value corresponding to the electrical stimulation signal, the method further comprises:
using the electrical stimulator to generate a first predetermined number of electrical stimulation signals, perform a buck operation on a second predetermined number of electrical stimulation signals in the first predetermined number of electrical stimulation signals, and sample the bucked electrical stimulation signals to calculate the total impedance value corresponding to the electrical stimulation signals.

16. A non-implantable electrical stimulation device, comprising:
an electrode assembly;
an electrical stimulator, wherein the electrical stimulator is detachably electrically connected to the electrode assembly, and the electrical stimulator comprises:
a storage unit, configured to store an impedance value of the electrical stimulator and an impedance value of the electrode assembly;
an electrical stimulation signal generating circuit, configured to generate an electrical stimulation signal, and use the electrical stimulation signal to perform electrical stimulation of a target area;
a sampling module, configured to sample the electrical stimulation signal; and
a calculation module, configured to calculate a total impedance value corresponding to the electrical stimulation signal according to the sampled electrical stimulation signal, and calculate a tissue impedance value according to the total impedance value, the impedance value of the electrical stimulator and the impedance value of the electrode assembly, wherein the tissue impedance value is used to calculate an energy value corresponding to the electrical stimulation signal transmitted to the target area.

17. The non-implantable electrical stimulation device as claimed in claim 16, wherein the electrode assembly comprises two electrodes.

18. The non-implantable electrical stimulation device as claimed in claim 17, wherein the two electrodes are thin film electrodes.

19. The non-implantable electrical stimulation device as claimed in claim 16, wherein the electrode assembly comprises a conductive gel.

20. The non-implantable electrical stimulation device as claimed in claim 16, wherein the target area comprises a skin of a living body.

21. The non-implantable electrical stimulation device as claimed in claim 16, wherein the electrical stimulation signal further comprises a plurality of pulse signals, and the electrical stimulator samples at least one of the plurality of pulse signals to calculate the total impedance value corresponding to the at least one of the plurality of pulse signals.

22. The non-implantable electrical stimulation device as claimed in claim 16, wherein when the energy value generated by the electrical stimulation signal generating circuit is accumulated to a target energy value, electrical stimulation is stopped performing in the target area.

23. The non-implantable electrical stimulation device as claimed in claim 16, wherein the impedance value of the electrical stimulator and the impedance value of the electrode assembly are measured in an environment of the same frequency as the electrical stimulation signal.

24. The non-implantable electrical stimulation device as claimed in claim 16, wherein the electrical stimulator is electrically connected to an external control device, the electrical stimulator transmits the tissue impedance value to the external control device, the electrical stimulator or the external control device determine whether the tissue impedance value is within a predetermined range, wherein in an electrical stimulation phase, when the tissue impedance value is outside the predetermined range, the electrical stimulator or the external control device instructs the electrical stimulator to stop an electrical stimulation of the electrical stimulation phase, and when the tissue impedance value is within the predetermined range, the electrical stimulator or the external control device instructs the electrical stimulator to continue the electrical stimulation of the electrical stimulation phase.

25. The non-implantable electrical stimulation device as claimed in claim 16, wherein in a non-electrical stimulation phase, the calculation module samples a current signal of the electrical stimulation signal to generate an average current value, and determines whether the average current value is greater than or equal to a predetermined current value, wherein when the average current value is less than the predetermined current value, the calculation module increases a voltage value of the electrical stimulation signal by a preset value, and samples the electrical stimulation signal again.

26. The non-implantable electrical stimulation device as claimed in claim 16, wherein a frequency of the electrical stimulation signal is between 1 KHz and 1 MHz.

27. The non-implantable electrical stimulation device as claimed in claim 24, wherein an upper limit value of the predetermined range is 2000 ohms, and a lower limit value of the predetermined range is 70 ohms.

28. The non-implantable electrical stimulation device as claimed in claim 16, wherein the electrical stimulator obtains a plurality of tissue impedance values, and the electrical stimulator calculates a tissue impedance average value of the plurality of tissue impedance values;
wherein the electrical stimulator determines whether the tissue impedance average value is greater than a previous tissue impedance average value, and whether a difference between the tissue impedance average value and the previous tissue impedance average value is greater than a first predetermined ratio, wherein when the tissue impedance average value is greater than the previous tissue impedance average value and the difference is greater than first the predetermined ratio, the tissue impedance average value and the previous tissue impedance average value are averaged to generate an average value, and an output tissue impedance average value is updated according to the average value, and wherein when the tissue impedance average value is not greater than the previous tissue impedance average value or the difference is not greater than first predetermined ratio, the output tissue impedance average value is updated according to the tissue impedance average value.

29. The non-implantable electrical stimulation device as claimed in claim 28, wherein the electrical stimulator determines whether a difference between the output tissue impedance average value and a previous output tissue impedance average value is greater than a second predetermined ratio, wherein when the difference is not greater than the second predetermined ratio, the external control device instructs that an output current is not adjusted, wherein when the difference is greater than the second predetermined ratio, the external control device further determines whether the output tissue impedance average value is less than a predetermined impedance value, wherein when the output tissue impedance average value is not less than the predetermined impedance value, the external control device instructs the electrical stimulator not to adjust the output current, and wherein when the output tissue impedance average value is less than the predetermined impedance value, the output current is adjusted according to the tissue impedance average value.

30. The non-implantable electrical stimulation device as claimed in claim 16, wherein the electrical stimulator is used to generate a first predetermined number of electrical stimulation signals, perform a buck operation on a second predetermined number of electrical stimulation signals in the first predetermined number of electrical stimulation signals, and sample the bucked electrical stimulation signals to calculate the total impedance value corresponding to the electrical stimulation signals.
